(19) United States
(12) Patent Application Publication
(10) Pub. No.: US 2005/0038491 A1
(43) Pub. Date: Feb. 17, 2005

(54) Cardiac pacing lead having dual fixation and method of using the same

(76) Inventor: Scott Graham Haack, Massillon, OH (US)

Correspondence Address: SAND & SEBOLT, AEGIS TOWER, SUITE 1100, 4940 MUNSON STREET, NW, CANTON, OH, 44718-3615, US

(21) Appl. No.: 10/915,211

(22) Filed: Aug. 10, 2004

Related U.S. Application Data
(60) Provisional application No. 60/494,435, filed on Aug. 11, 2003.

(51) Int. Cl. A61N 1/05

(52) U.S. Cl. 607/126; 607/127; 607/128

(57) ABSTRACT

A cardiac pacing lead for attaching a pulse generating device, such as a pacemaker or defibrillator to the heart, and a method for attaching and using the same. The cardiac pacing lead includes a passive fixation, such as tines for engaging trabecular tissue; and an active fixation, such as a helical screw, for engaging myocardial tissue. The active fixation is selectively movable between a first position where it lies within the flexible tube, and a second position where it extends at least partially from the tube at one end of the flexible tube and can be screwed into the heart tissue. The active fixation is only engaged once heart tissue with suitable conductivity properties is located by tests conducted through the electrically active tip of the active fixation. The cardiac pacing lead is secured to the patient's heart by providing an incision in the patient's body, threading the pacing lead through the circulatory system, locating suitable heart tissue by conducting tests via the active fixation, securing the passive fixation to the suitable heart tissue; securing the active fixation to the suitable heart tissue; generating an electrical pulse from a pulse generating device and passing the same through the conductor and active fixation into the patient's heart.

### CROSS REFERENCE TO RELATED APPLICATIONS

This application claims priority from U.S. Provisional Application Ser. No. 60/494,435 filed Aug. 11, 2003; the disclosure of which is incorporated herein by reference.

### BACKGROUND OF THE INVENTION

1. Technical Field

This invention generally relates to cardiac pacing leads for the attachment of pacemakers, defibrillators or other electrical stimulating devices to the heart. More particularly, the invention relates to a cardiac pacing lead that includes both an active and a passive fixation to the heart.

2. Background Information

In order to connect a pacemaker, defibrillator or other electrical stimulating device to the heart, it is necessary to connect a cardiac pacing lead to the myocardium or trabecular tissue of the heart. In the past, this has been done by using either an active fixation, such as a corkscrew type connector, or a passive fixation, such as a hook or tine type connector. A corkscrew type connector has a helical screw that punctures the myocardium and is then screwed into the myocardium to permanently fix the pacing lead to the heart. The surgeon conducts various tests through the pacing lead to determine if the heart tissue in that area of the heart has the necessary properties to effectively and safely conduct an electrical current to the heart. If the necessary properties are found in that tissue, the corkscrew is fulled twisted into the tissue and the lead is then connected to the electrical stimulating device. The problem with this type of active fixation is that when the corkscrew is screwed into the tissue to measure impedance, thresholds and the like, it tends to damage the tissue. If the tissue does not have the necessary properties, the corkscrew has to be withdrawn and be moved to a different location. The device may need to be moved several times in order to find tissue with the right properties. This not only leaves the heart with several areas that have been partially damaged, but it is also time consuming and may result in damage to the device itself. This problem has been partially addressed in the prior art by the provision of mechanisms to keep the helical screw retracted within the pacing lead, measuring the electrical transmission properties of the tissue with the tip of the lead and then extending the corkscrew once appropriate tissue has been located. This type of mechanism is disclosed in U.S. Pat. No. 6,381,500 B1 to Fischer, Sr. While the screw-type mechanism functions well to keep the pacing lead attached to the heart, there is a problem during the tissue testing and connecting phases. During the time between testing and connecting the screw, the physician has to physically maintain the position of the tip relative to the tested tissue. If he or she moves even slightly, the position of the tip may be shifted and the lead may consequently be connected to tissue that is less suitable for conducting an electrical current. It may therefore be necessary to remove the screw and shift the device to a new position in the heart. Furthermore, after installation of the pacing lead, the device may become detached from the heart tissue during normal activities on the part of the patient, causing damage to the heart tissue when it detaches from the same.

The other type of commonly used fixation is a passive fixation, namely a tine or hook type device which is typically used to connect a pacing lead to the trabecular tissue of the heart. This type of device has an advantage over the corkscrew type in that it allows for easier release and moving of the device if the tested tissue does not have the desired properties. Additionally, the tines do not tend to damage the trabecular tissue when they are pulled free. The tine type devices do, however, have a disadvantage in that they have a tendency to become dislodged during regular movement on the part of the patient. Additionally, if the electrical stimulation device is a defibrillator, the tines may also become dislodged through the action of the electric shocks delivered to the heart tissue through the electrode in the pacing lead. Dislodgement is most common in the first month or two after placement of the cardiac pacing lead. After this period, fibrous tissue tends to grow around the site of the implant and as it does so, it locks the lead in place. Even though tine-type devices may be more easily moved than corkscrew-type devices, they may be difficult to feed into the trabecular tissue because the tines extend outwardly from the tip of the lead. Additionally, the tines may become entangled among the fibers of the trabecular tissue and be damaged when the lead is pulled free to move it to a new location in the heart. When the device is then reattached in a new location, the tines may not engage the trabecular tissue as securely as before. This problem has been addressed in U.S. Pat. No. 4,913,164 to Greene et al. The device taught by Greene et al. is one in which the tines may be extended to engage the trabecular tissue or may be withdrawn so that the device can be moved to a different location in the heart. The Greene et al. device, however, still suffers from the potential risk of dislodgement once appropriate tissue has been located and the device has been installed.

There is therefore a need in the art for a cardiac pacing lead that may be easily moved from one location in the heart to another without causing damage to the heart tissue, but which has a reduced tendency to be dislodged from the tissue once an appropriate tissue site is located and the lead is connected to the heart tissue.

### BRIEF DESCRIPTION OF THE DRAWINGS

The preferred embodiment of the invention, illustrative of the best mode in which applicant has contemplated applying the principles, is set forth in the following description and is shown in the drawings and is particularly and distinctly pointed out and set forth in the appended claims.

FIG. 1 is a partial perspective view of a prior art cardiac pacing lead having passive fixation;

FIG. 2 is a partial perspective view of a prior art cardiac pacing lead having an active fixation;

FIG. 3 is a perspective view of a cardiac pacing lead in accordance with the present invention;

FIG. 4 is an enlarged partial perspective view of the connector end of the cardiac pacing lead of FIG. 3;

FIG. 5 is a partial cross-sectional front view of a patient's body with the cardiac pacing lead being connected to the heart;

FIG. 6 is an enlarged partial cross-sectional front view of a patient's heart showing the cardiac pacing lead connected to the heart tissue;

FIG. 7A is an enlarged partial cross-sectional front view of the heart showing the passive fixation of the cardiac pacing lead engaged with the heart tissue;

FIG. 7B is an enlarged partial cross-sectional front view of the heart showing both the active and passive fixation of the cardiac pacing lead engaged with the heart tissue;

FIG. 8 is an enlarged perspective view of the end of the cardiac pacing lead showing extension of the helical screw from within the housing of the passive fixation.

### DETAILED DESCRIPTION OF THE INVENTION

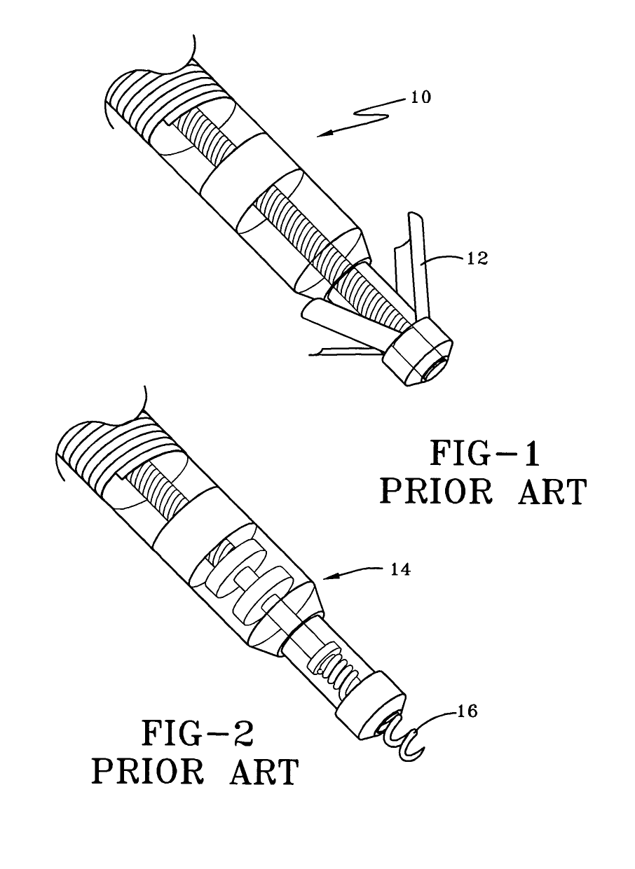

FIGS. 1 and 2 illustrate the cardiac pacing lead connections that have been disclosed in the prior art. FIG. 1 shows a first cardiac pacing lead 10 with a passive fixation in which a plurality of tines 12 are adapted to engage with trabecular tissue (not shown). FIG. 2 shows a second cardiac pacing lead 14 with an active fixation 14 in which a helical screw 16 is used to engage the myocardium (not shown).

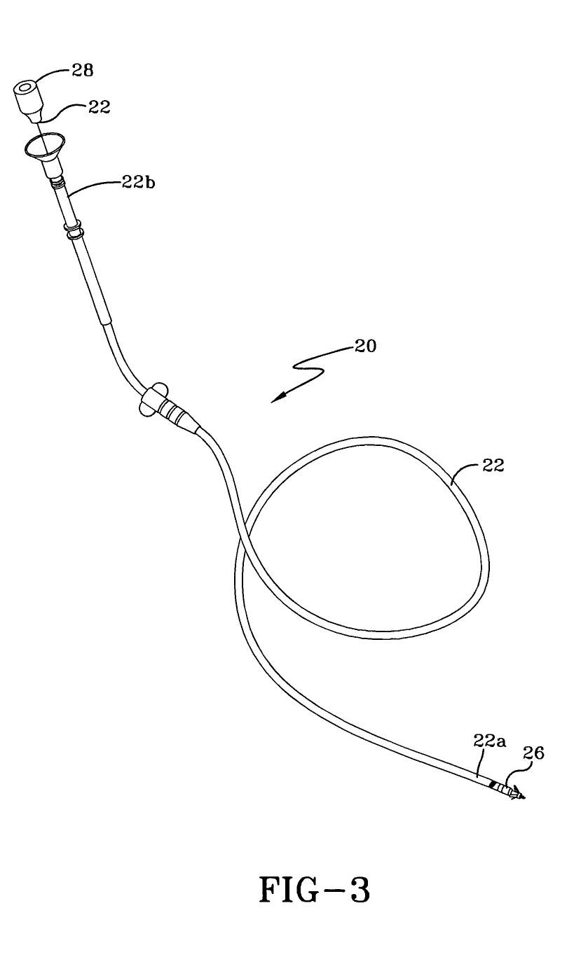

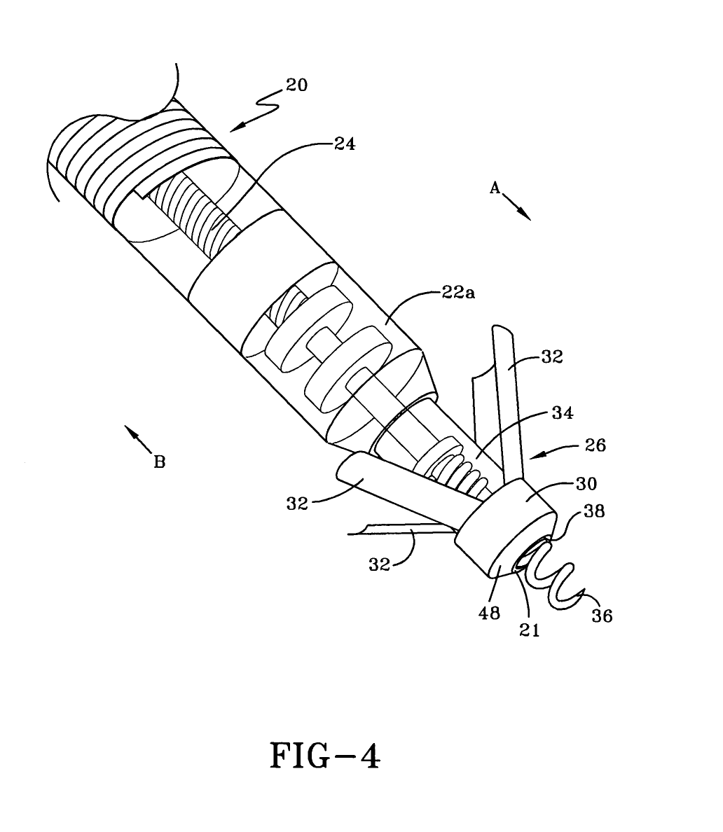

Referring to FIGS. 3 and 4 there is shown a cardiac pacing lead 20 in accordance with the present invention. The lead 20 includes a flexible tube 22 made of a suitable material such as a non active polyurethane and/or silicone. The tube 22 may be either straight or preformed into a suitable shape for implantation, such as a commonly used J-shape. A flexible conductor wire 24 is carried within tube 22. Conductor wire 24 may be either a straight or coiled wire. A connector 26 is disposed at one end 22a of tube 22 and a stylet 28 extends out of the other end 22b of tube 22. The physician uses stylet 28 to steer the lead 20.

Referring still to FIG. 4, there is shown an enlargement of the connector 26 of the present invention. Connector 26 includes both a passive fixation and an active fixation for engaging heart tissue. The passive fixation comprises a housing 30 having a plurality of tines 32 extending outwardly therefrom. Both housing 30 and tines 32 preferably are manufactured from a non active polyurethane or silicone material, but may also be manufactured from a flexible metal or a combination of metal and materials such as polyurethane. The forward end 34 of tube 22 is of a smaller diameter than the diameter of the remainder of tube 22. This facilitates the movement of tube 22 through the body of the patient, generally in the direction of arrow A. Housing 30 is slipped over forward end 34 so that tines 32 are flattened against forward end 34 as tube 22 is moved through the patient's venous system. In order to engage tines 32 with the trabecular tissue of heart, tube 22 must be pulled slightly rearwardly in the direction of arrow B (FIG. 4). This movement causes tines 32 to be spread apart and the tines 32 catch the fibers of the trabecular tissue thereby preventing connector 26 from being easily withdrawn from the heart. Alternatively, tines 32 may be connected to a mechanism (not shown) that enables the physician to extend or retract the tines 32. Mechanisms for extending and retracting the tines are known in the prior art.

An active fixation is also provided. The active fixation comprises a helical screw 36 that is connected to the conductor wire 24 at one end and is adapted to extend through an aperture 38 in housing 30. Helical screw 36 is movable from a first retracted position where it is enclosed within forward end 34, to a second extended position where it projects at least partially from housing 30. Mechanisms for extending and retracting a helical screw within a pacing lead are well known in the prior art.

Lead 20 has an electrically active tip in that helical screw 36 is connected to conductor wire 24. Whether screw 36 is withdrawn into forward end 34 or extends outwardly from forward end 34 of housing 30, screw 36 may be utilized to conduct the electrical testing of the heart tissue. An electrically active ring electrode 21 may also be provided to assist in making electrical contact with the heart tissue. Once screw 36 is engaged in the heart tissue, current is able to flow through screw 36 and, if provided, ring electrode 21, thereby providing a secure, grounded electrical connection to the heart.

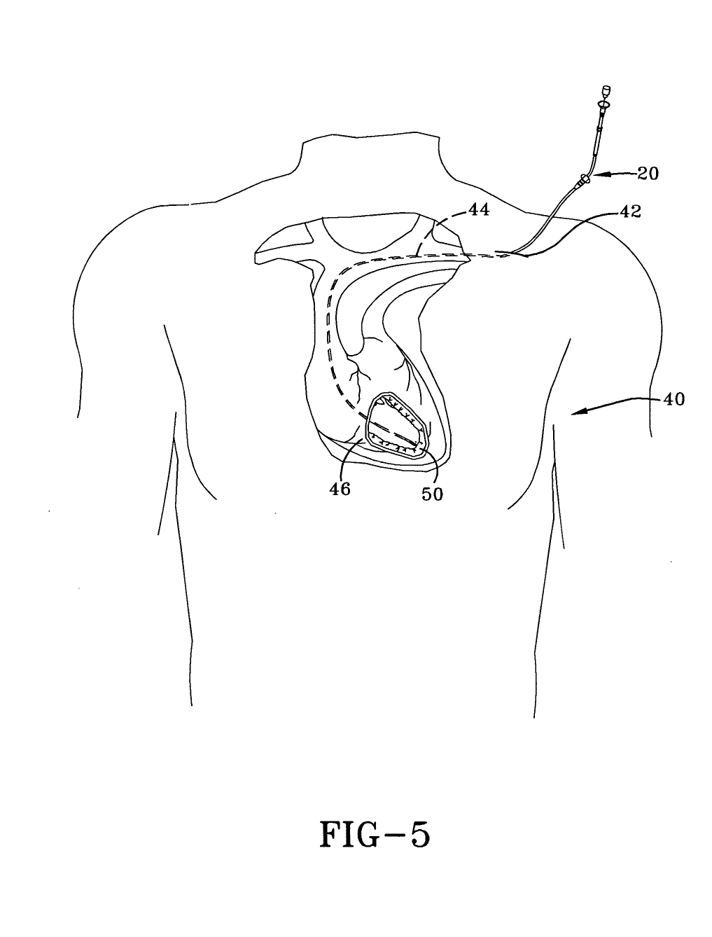

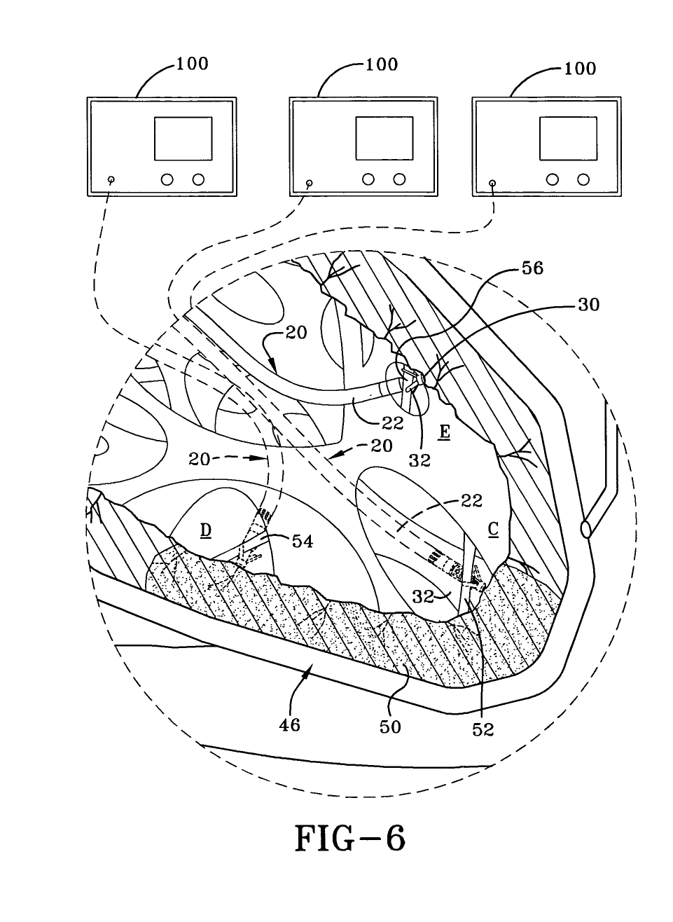

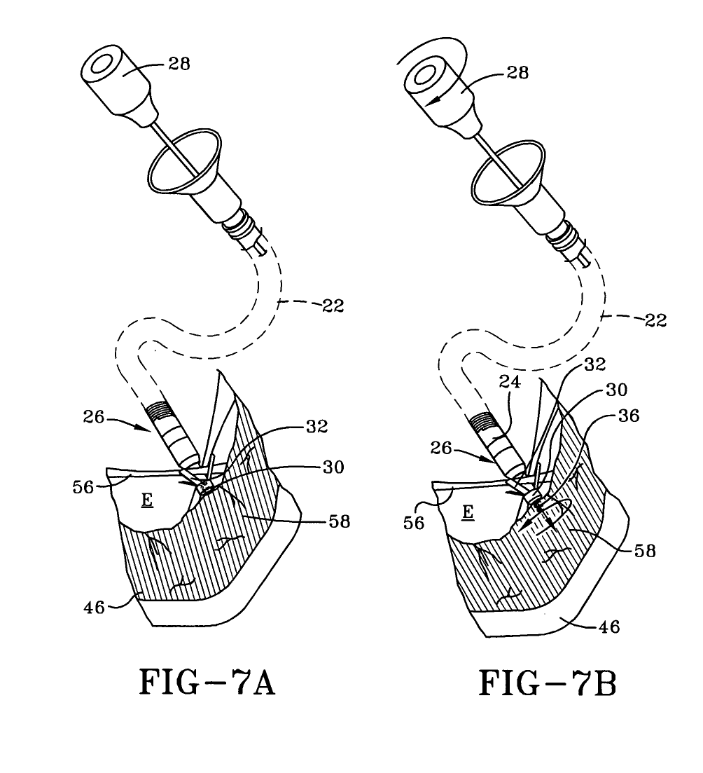

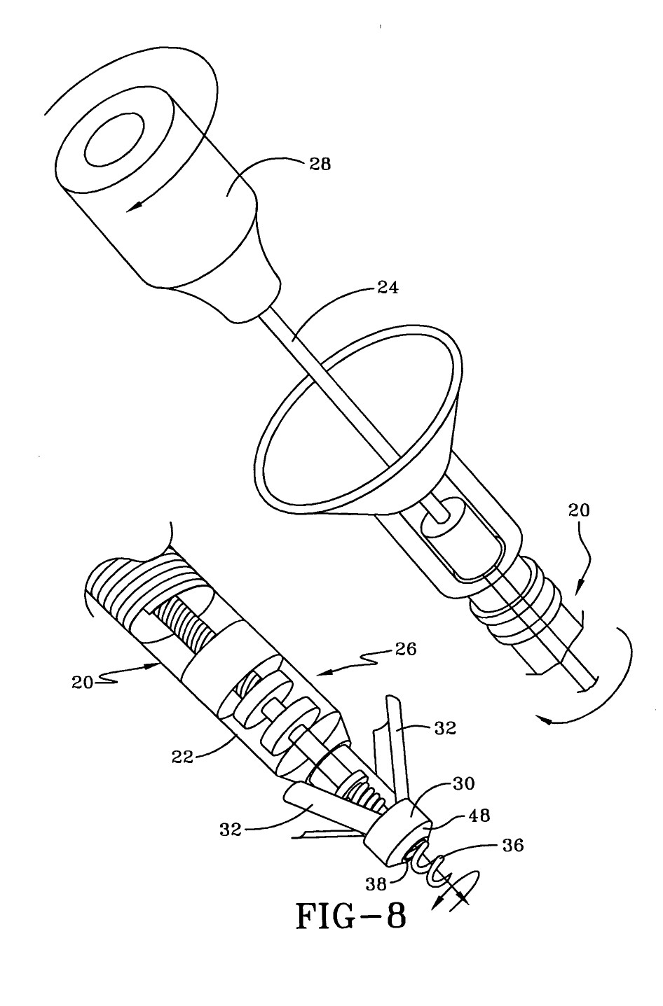

Referring to FIGS. 5-8, when lead 20 is to be inserted into a patient's body 40, a small incision 42 is made, usually in the mid-clavicular line just below the clavicle. The lead 20 at this point has helical screw 36 retracted within forward end 34 and, if provided, the mechanism for maintaining tines 32 against forward end 34 is engaged. Lead 20 is then inserted into a vein and the physician guides the lead 20 through the venous system 44 using a fluroscopy machine. As lead 20 moves through the venous system 44, tines 32 lie against forward end 34 and they therefore do not interfere with the travel of lead 20 through system 44. Lead 20 is fed through the venous system 44 until it reaches the heart 46 and the tip 48 of housing 30 engages heart tissue 50 in an area C (FIG. 6) of the heart. At this area C, the physician pulls the lead 20 rearwardly (in the direction of arrow B of FIG. 4) or, alternatively, engages the mechanism to open or spread tines 32. The tines 32 spread outwardly from housing 30 and are entrapped in the fibers of the trabecular tissue 52 of heart 46. The physician connects lead 20 to devices 100 that allow him/her to conduct the standard tests, such as impedance tests, to determine if area C has the necessary physical properties to conduct electrical signals to the heart 46. If the tissue in area C proves to be unsuitable, then, if provided, the mechanism to retract tines 32 is engaged and lead 22 is moved to area D of heart 46. If a retracting mechanism is not provided, the physician pulls the lead 20 gently in the direction of arrow B (FIG. 4) until tines 32 disengage from the trabecular tissue 52 and moves the lead 20 until tip 48 engages area D. The tines 32 are then redeployed to engage the fibers of the trabecular tissue 54 in area D. The standard tests are then conducted again to determine if the tissue in area D has the appropriate physical properties. If the tissue is again found to be unsuitable, the physician moves the lead 20 yet again to area E of the heart 46 in the previously described manner. The tines 32 are engaged with the trabecular tissue 56 in the aforementioned manner. Tests are again run to determine whether the tissue in area E has the appropriate properties. If the tissue in area E is found to be suitable, the physician then engages conductor wire 24 with an appropriate tool (not shown) which allows the physician to extend or retract screw 36 by moving the conductor wire 24 in either a clockwise or counterclockwise direction respectively as is shown in FIG. 7B-8. These types of tools are known in the prior art. Any other suitable mechanism for extending the screw 36 may alternatively be utilized. Screw 36 is turned so that it projects through aperture 38 in housing 30 and is then screwed into the myocardial tissue in area E.

The conductor wire 24 is then electrically connected to the pulse generator or electrical stimulation device (not shown) in a conventional manner and the stimulation device is implanted into the patient's body 40. Because the connector 26 has both a passive and active fixation via the tines 32 and helical screw 36, it tends to not be easily dislodged by the patient's normal movements. Additionally, because the helical screw 36 is only engaged when an appropriate area E of myocardium is located, the damage to the heart 46 caused by multiple rounds of extending and retracting of the helical screw 36 is negated. The one time extension of helical screw 36 also preserves the life and electrical conductivity of screw 36 as multiple extensions and retractions of the screw 36 tend to cause damage to the screw 36 itself. Because the passive fixation, i.e., the tines 32, maintains the position of the lead 20 within the heart 46, the physician can have confidence that the screw 36 is being inserted into the tested tissue. Additionally, the combination of the active and passive fixations of the screw 36 and tines 32 also tends to reduce the necessity for repeat implantation of the lead 20 because of dislodgement of lead 20 through either the patient's movements, or a shock delivered by a defibrillator. Furthermore, because lead 20 is firmly attached to the heart tissue, the electric current flowing from the electrical stimulating device through screw 36, tends to be delivered to the heart 46 more efficiently.

While the passive fixation of the present invention is shown to be a plurality of tines 32 integrally formed with a housing 30, it will be understood by those skilled in the art that other passive fixations, such as hooks, may also be used in combination with an active fixation such as a helical screw without departing from the scope of the present invention. Furthermore, while the above description indicates that the helical screw is the electrically active fixation and the tines are non-conductive, it will be understood that the tines could comprise the electrically active fixation and the helical screw could be non-conductive.

In the foregoing description, certain terms have been used for brevity, clearness, and understanding. No unnecessary limitations are to be implied therefrom beyond the requirement of the prior art because such terms are used for descriptive purposes and are intended to be broadly construed.

Moreover, the description and illustration of the invention is an example and the invention is not limited to the exact details shown or described.

1. A cardiac pacing lead comprising:
a flexible tube;
a conductor disposed within the flexible tube; the conductor being adapted to be connected to a pulse generating device;
a passive fixation carried by one of the flexible tube and conductor; and
an active fixation carried by the other of the flexible tube and the conductor.

2. The cardiac pacing lead as defined in claim 1, wherein the passive fixation comprises a housing having a plurality of tines extending outwardly therefrom.

3. The cardiac pacing lead as defined in claim 2, wherein the housing has a forward end and the tines radiate outwardly away from the forward end of the housing and toward a rear end of the flexible tube.

4. The cardiac pacing lead as defined in claim 3, wherein the tines are selectively movable between a retracted position where they lie against the tube and an extended position where they lie a spaced distance from the tube.

5. The cardiac pacing lead as defined in claim 4, wherein the forward end of the housing defines an aperture and the active fixation extends through the aperture.

6. The cardiac pacing lead as defined in claim 5, wherein the active fixation is movable between a first position where lies within the tube and a second position where it extends through the aperture and projects out of the tube.

7. The cardiac pacing lead as defined in claim 6, wherein the active fixation is a helical screw.

8. The cardiac pacing lead as defined in claim 7, wherein the passive fixation is manufactured from one of non-reactive polyurethane and silicone.

9. The cardiac pacing lead as defined in claim 8, wherein the active fixation is manufactured from metal.

10. The cardiac pacing lead as defined in claim 9, wherein the active fixation is electrically conductive.

11. The cardiac pacing lead as defined in claim 1, wherein the passive fixation is a hook mechanism.

12. The cardiac pacing lead as defined in claim 11, wherein the active fixation is a helical screw.

13. The cardiac pacing lead of claim 12, wherein the helical screw is movable between a first position where the screw is retracted into the tube and a second position where the screw is extended at least partially from the tube.

14. The cardiac pacing lead of claim 1, further comprising a stylet connected to the conductor, the stylet being manipulable to move the conductor within the flexible tube and thereby move the active fixation.

15. A method of attaching a cardiac pacing lead to the heart and using the same comprising the steps of:
providing a cardiac pacing lead comprising a flexible tube and a conductor disposed therein and having a passive fixation and an active fixation;
providing a pulse generating device;
making an incision in the patient's body;
threading the pacing lead through the patient's circulatory system to the heart;
conducting tests on the tissue of the heart to locate suitable tissue for operation of the pacing lead;
securing the pacing lead to the heart tissue via the passive fixation;
securing the pacing lead to the heart tissue via the active fixation;
generating a pulse from the pulse generating device.

16. The method as defined in claim 15, wherein step of providing a cardiac pacing lead includes providing a pacing lead where the passive fixation has a plurality of tines extending outwardly therefrom.

17. The method as defined in claim 16, wherein the tines are movable between a first position where they lie in abutting contact with an outer surface of the flexible tube and a second position where they lie a spaced distance from the outer surface of the tube; and the step of threading the pacing lead includes keeping the tines in abutting contact with the outer surface of the tube.

18. The method as defined in claim 17, wherein the step of securing the passive fixation to the heart includes the steps of:
moving the tines to the second position where they lay a spaced distance from the outer surface of the tube; and
puling the pacing lead in the opposite direction to the direction in which it was threaded through the circulatory system to engage the tines in the heart tissue.

19. The method as defined in claim 18, wherein the step of providing a cardiac pacing lead includes providing a pacing lead having an active fixation which is movable between a first position where the active fixation is contained within the flexible tube and a second position where the active fixation extends at least partially from the tube; and wherein the step of threading the pacing lead through the circulatory system includes the step of keeping the active fixation in the second position.

20. The method as defined in claim 19, wherein the step of securing the active fixation to the heart tissue includes the step of moving the active fixation to the second position wherein the active fixation extends outwardly from the tube to engage the heart tissue;

21. The method as defined in claim 20, wherein the active fixation is a helical screw and the step of securing the active fixation to the heart tissue includes the step of screwing the helical screw into the heart tissue.

22. The method as defined in claim 21, wherein the step of screwing the helical screw into the heart tissue includes the steps of:
providing a stylet connected to the cardiac pacing lead;
manipulating the stylet to screw the helical screw into the heart tissue.

23. The method as defined in claim 16 wherein the step of providing a cardiac pacing lead includes the step of providing an electrically conductive active fixation.

24. The method as defined in claim 23, wherein the step of providing a cardiac pacing lead includes the step of providing a non-conductive passive fixation.
